(19) United States
(12) Patent Application Publication
(10) Pub. No.: US 2006/0206992 A1
(43) Pub. Date: Sep. 21, 2006

(54) Multipart tool belt

(75) Inventors: Donald E. Godshaw, Evanston, IL (US); Andrezj M. Redzisz, Wheeling, IL (US)

Correspondence Address: BANNER & WITCOFF, LTD., TEN SOUTH WACKER DRIVE, SUITE 3000, CHICAGO, IL, 60606, US

(73) Assignee: Travel Caddy, Inc. d/b/a Travelon, Elk Grove Village, IL (US)

(21) Appl. No.: 11/415,813

(22) Filed: May 2, 2006

Related U.S. Application Data
(63) Continuation-in-part of application No. 11/125,711, filed on May 10, 2005.
Application No. 11/125,711 is a continuation-in-part of application No. 10/813,524, filed on Mar. 30, 2004, now Pat. No. 7,011,241.
Application No. 10/813,524 is a continuation of application No. 10/339,902, filed on Jan. 10, 2003, now Pat. No. 6,712,251.
Application No. 10/339,902 is a division of application No. 09/921,125, filed on Aug. 2, 2001, now abandoned.
Application No. 09/921,125 is a continuation-in-part of application No. 09/359,339, filed on Jul. 21, 1999, now Pat. No. 6,390,348.
(60) Provisional application No. 60/222,713, filed on Aug. 3, 2000.

(51) Int. Cl. A61F 5/00; A41F 9/00; A41F 15/00

(52) U.S. Cl. 2/338; 2/44; 602/19; 224/904

(57) ABSTRACT

A multipart tool belt includes a central segment and adjustable length lateral side segments which may be joined together to provide a customized belt for use as part of a tool belt assembly.

### CROSS REFERENCE TO RELATED APPLICATIONS

This is a continuation-in-part of Ser. No. 11/125,711 filed May 10, 2005 for “Tool Belt Carrier and Pouch Constructions” which is a continuation-in-part of Ser. No. 10/813,524 filed Mar. 30, 2004, for “Tool Belt Carrier, and Pouch Constructions”, which is a continuation-in-part of Ser. No. 10/339,902 filed Jan. 10, 2003 for “Low Slung Tool Carrier”, now U.S. Pat. No. 6,712,251 issued Mar. 30, 2004, which is a division of Ser. No. 09/921,125 filed Aug. 2, 2001 for “Low Slung Tool Carrier” which is a continuation-in-part of Ser. No. 09/359,339 filed Jul. 21, 1999 for “Tool Belt”, now U.S. Pat. No. 6,390,348, and provisional application Ser. No. 60/222,713 filed Aug. 3, 2000 for “Low Slung Tool Carrier” all of which are incorporated herewith by reference and for which priority is claimed.

### BACKGROUND OF THE INVENTION

In a principal aspect the present invention relates to a tool belt and, more particularly, to a tool belt which is adjustable to accommodate various girth sizes.

Construction workers, tradesman and the like typically use a tool belt attached about their waist to transport and maintain tools at a work site. Such belts are often fabricated from canvas and/or leather and may include a number of attached pockets or pouches which are designed to hold tools such as pliers, screwdrivers and the like. Desirable characteristics for such belts are durability and the capability to hold and store many tools and other items. The belts must also be comfortable and yet durable in order to withstand rugged circumstances. Additionally, such a belt must be designed to accommodate various types of tools and if possible permit alteration and adjustment in order to accommodate various types of tools, various sizes of tools and various sizes of workmen. Thus, there has remained a need for an improved tool belt which is comfortable to wear, rugged, economical and easily adapted for multiple uses.

### SUMMARY OF THE INVENTION

Briefly, the present invention is related to the inventions set forth in the cross reference designations and comprises an embodiment of a tool belt which includes at least two and preferably three separate and separable belt segments; namely, a left hand belt segment, a right hand belt segment and a center belt segment constructed to connect the left hand and right hand segments. The left hand and right hand segments each include a number of sectors one or more of which may be physically removed to thereby adjust the length of the segments. Once the segments are appropriately customized in terms of their length, they are combined or joined with the center segment to provide a customized total length tool belt designed to fit around the girth of a tradesman or worker. A belt strap with a connecting buckle fits through loops of the belt segments and pouches may be attached to the belt strap or to the belt segments. As additional features, the belt segments are configured to provide cushioned areas that fit against the hips of the worker or tradesman and may also include ventilated or air flow passages to further facilitate the comfort of the assembled belt. In a preferred embodiment, the left hand and right hand segments are attachable to the center segment by means of hook and loop fasteners. Padding may be incorporated into the various segments to facilitate the comfort of the user of the belt construction.

Thus, it is an object of the invention to provide a tool belt construction which may be customized in length to accommodate the unique girth of a worker or tradesman.

Another object of the invention is to provide a belt construction which is comprised of multiple component segments that may be joined together in combination with a belt strap and pouches for the transport of worker tools.

Another object of the invention is to provide a tool belt construction and carrier comprised of a flexible, yet rugged material such as leather, canvas or other flexible fabric or similar materials.

Another object of the invention is to provide a tool belt construction which includes padding placed or positioned at strategic parts of the tool belt and, more particularly, of the segments comprising the belt construction to enhance the comfort and utility of the belt assembly or construction.

These and other objects, advantages and features of the invention will be set forth in the detailed description which follows.

### BRIEF DESCRIPTION OF THE DRAWING

In the detailed description which follows, reference will be made to the drawing comprised of the following figures:

FIG. 1 is a front plan view of the tool belt assembly of the invention in a first embodiment comprised of a left hand segment, a right hand segment and a connecting central segment;

FIG. 2 is a backside view of the assembly of FIG. 1;

FIG. 3 is a partially deconstructed view of the assembly of FIG. 1;

FIG. 4 is a front plan view of the component parts of the assembly of FIG. 1 rearranged for measurement of the girth of a worker during the customization of the assembly;

FIG. 5 is a plan view of a further step in the process of the customization of the assembly of FIG. 1;

FIG. 5A is a plan view of yet a further step of the assembly of the customized length belt segments;

FIG. 6 is an exploded plan view of the assembly of FIG. 1;

FIG. 7 is an exploded plan view of an alternative embodiment of an assembly of a left hand segment, a right hand segment and a central segment of a belt assembly;

FIG. 8 is an assembled front plan view of the assembly of FIG. 7;

FIG. 9 is an exploded perspective view illustrating the component layers of material incorporated within a belt segment of the type depicted in the assembly of FIGS. 1 and/or 7;

FIG. 10 is a plan view of a belt construction depicting a waist belt segment incorporating layers of the subassembly depicted in FIG. 9; and

FIG. 11 is a back or opposite side view of the belt assembly of FIG. 10.

### DESCRIPTION OF PREFERRED EMBODIMENTS

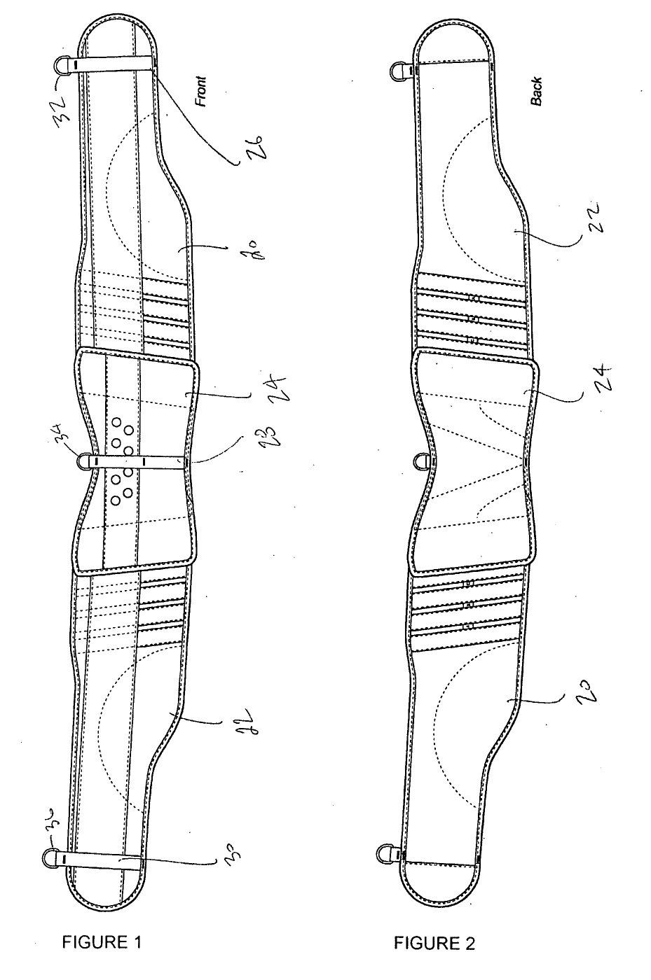

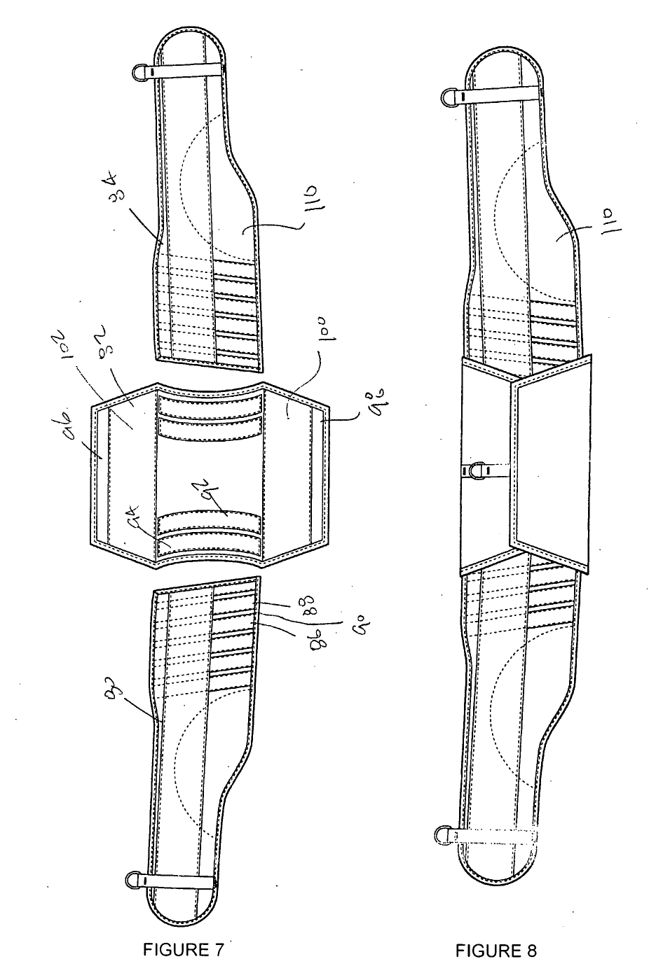

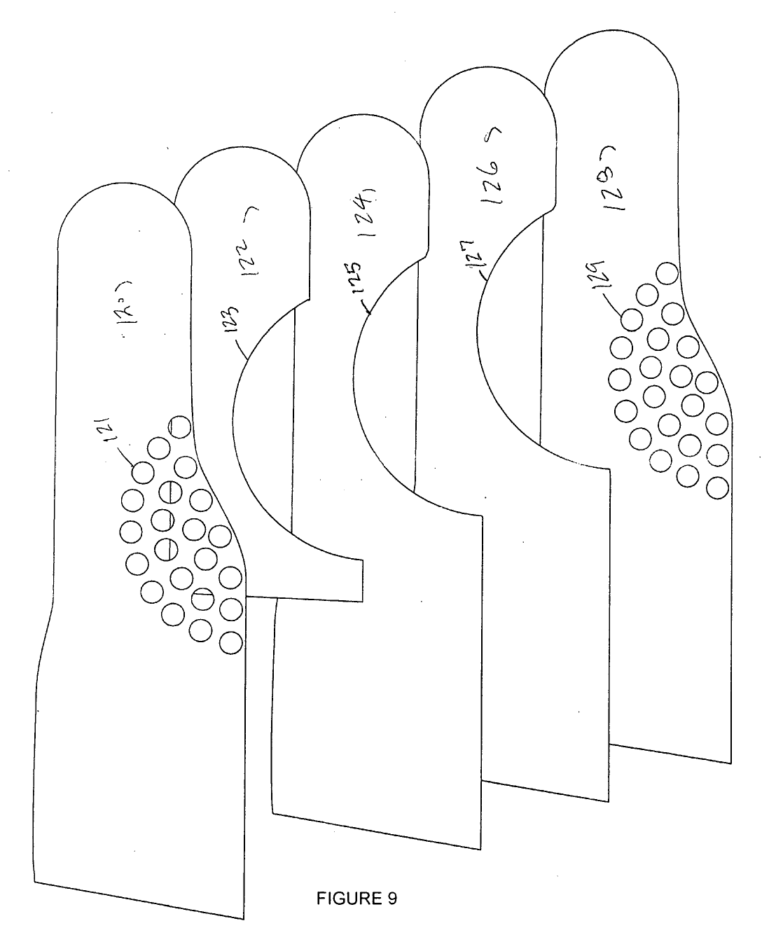

FIGS. 1-6 illustrate a first embodiment of the invention. FIGS. 7 and 8 illustrate a second embodiment. FIGS. 9-11 illustrate a feature of tool belt segments associated with the first and second embodiments which may be incorporated into those tool belt segments or into any tool belt as a feature to enhance comfort during use of the belt. Thus, the following description will be directed to a first embodiment, a second embodiment, and a feature which may be incorporated in either embodiment or in other tool belt assemblies.

The First Embodiment

The first embodiment of a multiple component tool belt includes a right hand segment 20, a left hand segment 22 and a connecting center segment 24. Referring to FIG. 1, there is depicted a plan view of the connected segments 20, 22 and 24 as viewed from a front or outside view of the component parts in assembled condition. Thus, the component parts or segments 20, 22, and 24 are joined together to provide an elongate belt assembly of desirable, unique length associated with the girth of a worker. The assembly includes a belt loop 26 associated with the right hand segment, a center belt loop 28 associated with a center section or segment 24 and a left hand belt loop 30 associated with the left hand segment 22 of the assembly. Thus, when the segments 20, 22 and 24 are arranged in an assembled unique, length condition, a belt strap with a connecting buckle (not shown) may be fitted through the loops 26, 28, 30 and fastened about the waist of a worker or tradesman to hold the multiple component tool belt assembly about the waist of that worker or tradesman.

Pouches, tool carrying elements and the like may be attached to the belt strap (not shown) as depicted, for example, in the cross referenced applications. Thus, multiple types of pouches, tool belt straps with buckles on the ends for fastening and the like may be used in combination with the multiple component assembly depicted in FIG. 1 and FIGS. 2-5.

Metal loops, such as loop 32, loop 34 and loop 36 may be fastened to suspenders or other supports again, as depicted, for example, in the cross referenced applications incorporated herewith by reference and for which priority is claimed. The present invention thus relates to the multiple component assembly depicted in the figures which is used in conjunction with a buckled belt strap and pouches and suspenders chosen by the tradesman or worker who is using the belt.

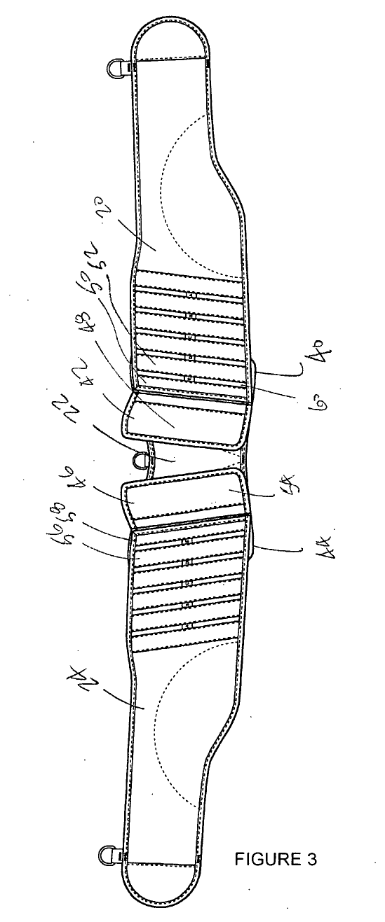

FIG. 2 shows the inside or backside of the assembly of FIG. 1. Thus, the central segment 24 connects the left hand segment 22 to the right hand segment 20. FIG. 3 is a partially disassembled version of the assembly depicted in FIG. 1. Thus, the right hand segment 20 is coupled to the left hand segment 22 by means of the central segment 24. The central segment 24 includes an inner flap 40 and an outer flap 42 for attachment to the right hand segment 20. An inner flap 44 and an outer flap 46 are provided for attachment to the left hand segment 22. The flaps 40, 42, 44, and 46 each include hook and loop fastener material, for example, material 48 for flap 42 on the surface thereof for cooperation with and engagement with hook and loop fastener material, such as hook and loop fastener material 50 and/or 52, on the surface of the right hand segment 20. Similarly, hook and loop fastener material 54 is provided for engagement with hook and loop fastener material 56 or 58 of the left hand segment 22. The flaps 40 and 44 likewise include cooperative hook and loop fastening material on their inside surfaces for engaging hook and loop fastening material on the opposed surface of the left and right hand segments 20 and 22. In this manner, the central segment 24 of the assembly may be attached and/or detached from the right hand segment 20 and the left hand segment 22.

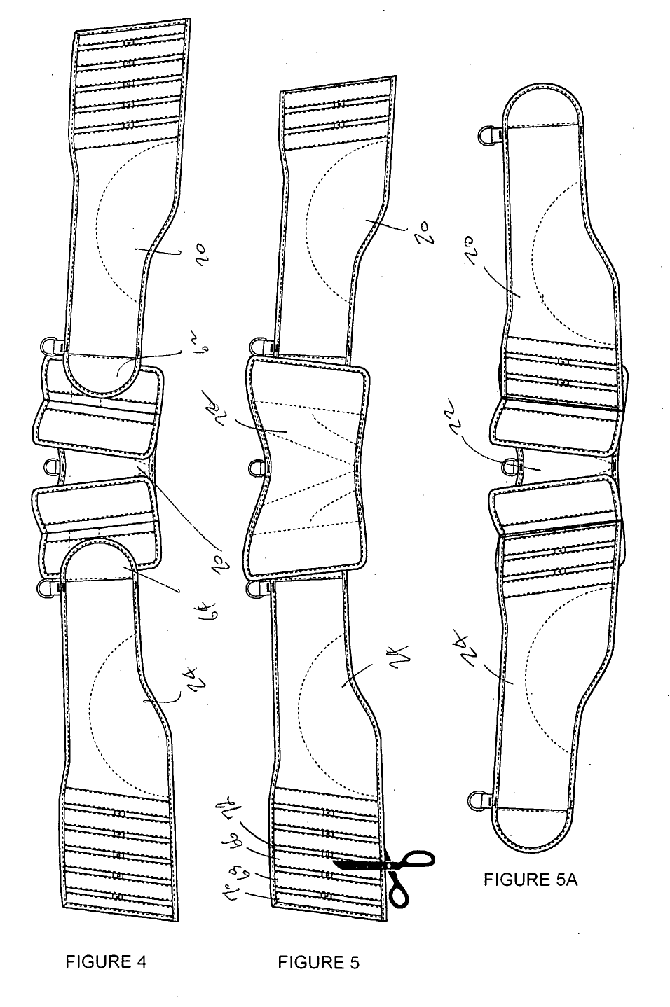

It is to be noted referring to FIGS. 4, 5 and 5A, as well as the other figures, that the left and right hand segments 20 and 22 include a multiplicity of separate generally parallel hook and loop fastener sectors or strips such as sectors 50 and 52 spaced longitudinally along the length of the segments 20 as well as a similar arrangement along the segments 22. The hook and loop fastener material, such as the strip thereof indicated as strip 50, is separated by a boundary 60 from the strip 52. In practice then, and in order to accommodate the girth of an individual using the assembly depicted, the central section or central segment 24 as depicted in FIG. 4 will be engaged with the outside end 62 of the right hand segment 20 and the outside end 64 of the left hand segment 24. The worker will then place the assembled segments about his girth after the central section 24 is attached by engagement of the hook and loop fastening material associated with the central segment and with the end sections 62 and 64 as depicted in FIG. 5. The strips of material that are unnecessary in order to provide appropriate fitting about the girth of an individual may then be removed, for example, by cutting as depicted in FIG. 5. Thus, hook and loop fastener strips 66, 68 and 70 are separated from the segment 24 by cutting along the boundary line 72. It will be noted that the right hand segment 20 already is depicted as having a similar number of segments or strips of the hook and loop fastener material removed. The ends of the right hand segment 20 and left hand segment 22 are then removed from the central segment 24 and the assembly is then reassembled as depicted in FIG. 5A to a customized length. A belt strap may then be fitted through the loops, for example, loops 26, 28 and 30 to provide a customized length tool belt assembly. Pouches, suspenders and the like may then be attached to the assembly.

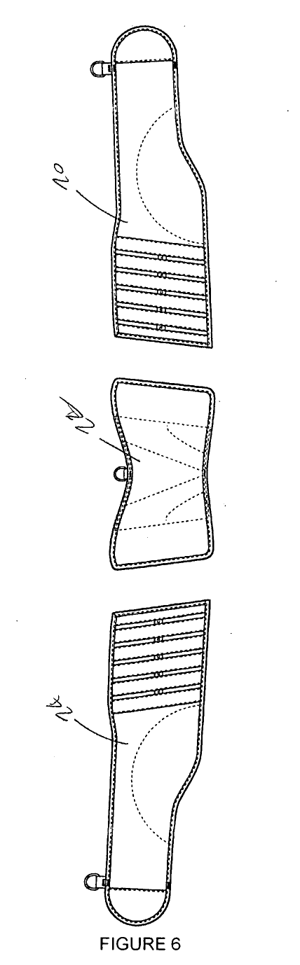

FIG. 6 further illustrates the component parts of the assembly; namely, the left hand segment 20, the central segment 24 and the left hand segment 22. These individual segments may then be customized to provide a definitive longitudinal length desirable for use by a worker or tradesman.

The Second Embodiment

FIGS. 7 and 8 illustrate a second embodiment of the invention. Referring to those figures a left hand segment 80 cooperates with a central segment 82 and a right hand segment 84 similarly cooperates with central segment 82. The left hand segment 80 includes a series of hook and loop fastener strips 86 and 88 which are separated by a boundary 90 for example. A desirable number of strips may be removed as previously explained. The remaining strips will be cooperative with strips associated with the central segment 82; namely, strips 92 and 94. The central segment 82 includes an upper flap 96 and a lower flap 98 which fold over the attached ends of the left hand segment 80 and right hand segment 84. The right hand segment 84 is likewise customized in length to accommodate the girth and longitudinal dimension of the assembly. Hook and loop fastener material 100 is provided for the flap 98 and similar hook and loop fastener material 102 is provided for the flap 96 so that when the flaps 96, 98 fold over they will engage with the strips of hook and loop fastener material that remain unremoved from the left and right hand segments 80 and 84. Thus, the embodiment depicted in FIGS. 7 and 8 is likewise capable of being customized in length to accommodate the desires of a worker or tradesman.

Additional Features

FIGS. 9, 10 and 11 illustrate an additional feature that is incorporated in embodiments of the invention. It will be noted, for example in FIGS. 7 and 8, that there is a region known as a hip support region for the right hand segment 84; namely, hip support region 110. This hip support region 110 is a region defined between an outer layer and an inner layer of fabric material such as canvas, leather or the like wherein cushion or padding material is omitted from the construction.

Referring to FIG. 9 the layers of material that are utilized to fabricate the segment 84 in a typical assembly are indicated. An outer layer of material; namely, outer layer 120 fits over a layer of cushion material 122 which, in turn, fits over a secondary layer of cushion or fabric material 124 which, in turn, fits over a further layer of cushion or fabric material 126 and all layers are attached to an outer layer 128. The layers 120, 122, 124, 126 and 128 are thus assembled or overlay one another to provide structural integrity yet remain adequately flexible for the support required for the belt segment 84.

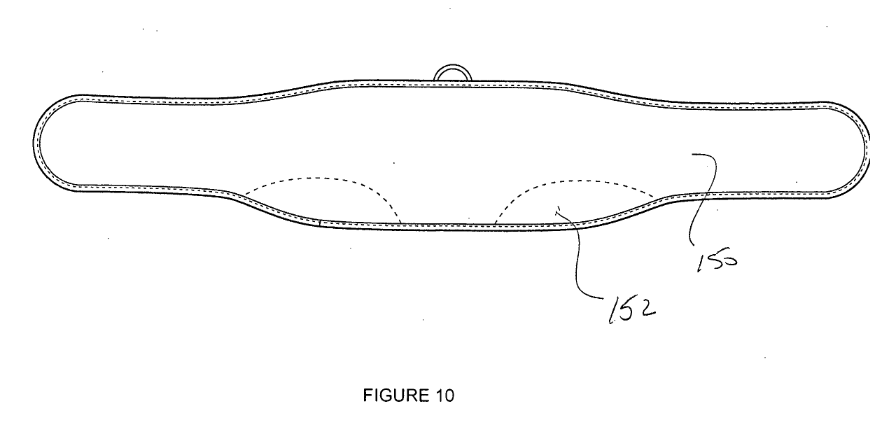

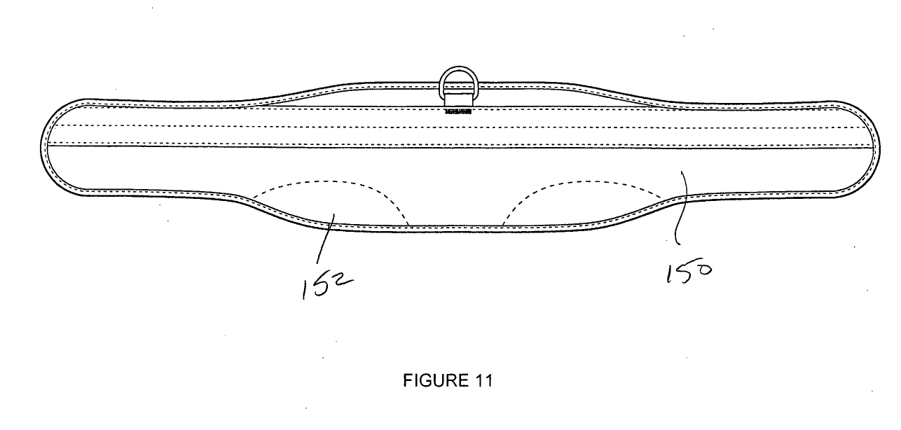

Each of the interior segments 122, 124 and 126 include an arcuate cut out section 123, 125 and 127, respectively. Those cut out sections are opposed by ventilating passages 121 and 129 associated with the outside layers 120 and 128, respectively, of the belt segment 84. In this manner, the arcuate segment 110 is thereby formed with air flow passages and with the cushioning material removed therefrom. However, the arcuate section is surrounded by cushion material. This construction enables placement of the segment 84 appropriately over the hip of an individual so that air flow will be available for comfort and so that there will be no excess material resting on the hip, but in a manner whereby the hip will support more effectively the segment 84 and thus the belt assembly and tools thereon. The utilization of this arcuate arrangement may be incorporated in the segments such as depicted with respect to the embodiments of FIGS. 1-5 or the embodiment of FIGS. 7 and 8 or alternatively, in a tool belt which is not comprised of multiple segments, for example, as depicted in FIGS. 10 and 11. Thus, referring to FIGS. 10 and 11, an arcuate portion 152 of the tool belt 150 has a construction as depicted in those figures. In sum, the arrangement depicted in FIGS. 9 may be included in any multiple part tool belt assembly or in a single waist belt.

Various other modifications of the embodiments depicted may be adopted. The size and arrangement and location of the hook and loop fastener materials may be varied. Alternative fastening materials other than hook and loop fastener materials may be utilized. The configuration of the various segments such as the right hand segment, the left hand segment and the central segment may be altered. The length of the various segments may be altered. That is, the central segment may be much longer and may incorporate the arcuate hip sections depicted. Thus, the invention is to be limited only by the following claims and equivalents.

What is claimed is:

1. A multiple component tool belt comprising, in combination:
a left hand belt segment including a buckle end, an opposite center connection end, and a lower edge margin, said center connection end including at least two adjacent end sectors separated by a boundary, one of said end sectors separable from the other along the boundary by detachment from the left hand belt segment;
a right hand belt segment including a buckle end, an opposite center connection end, and a lower edge margin, said right hand segment center connection end including at least two adjacent end sectors separated by a boundary with one of said end sectors separable from the other along the boundary by detachment from the right hand belt segment; and
a center belt segment attachable simultaneously to the left hand belt segment and the right hand belt segment at the respective center connection ends thereof to form an adjustable length tool belt comprised of the connected left hand segment, center segment and right hand segment characterized by a length dependent upon the number of end sectors maintained on the left hand and right hand segments.

2. The tool belt of claim 1 wherein the left hand and right hand segments are symmetrical about an axis through the center segment.

3. The tool belt of claim 1 wherein the center segment includes a hook and loop fastener for attachment to the left hand and right hand segments.

4. The tool belt of claim 1 wherein the center segment is comprised of a left hand pair of flaps and a right hand pair of flaps, attachable respectively to the left hand and right hand end sectors by fitting over the left and right hand end sectors.

5. The tool belt of claim 4 including a hook and loop fastener for attaching the center segment to at least one side segment.

6. The tool belt of claim 1 wherein the left hand segment includes more than two serially adjacent sectors, each sector separated from the adjacent sectors by a boundary and detachable along the boundary.

7. The tool belt of claim 1 wherein the right hand segment includes more than two serially adjacent sectors, each sector separated from the adjacent sectors by a boundary and detachable along the boundary.

8. The tool belt of claim 1 wherein detachment of a sector is effected by cutting the segment along a boundary.

9. The tool belt of claim 1 wherein the sectors are generally equally sized and configured.

10. The tool belt of claim 1 wherein the center segment comprises a median panel having a lower edge, an upper edge, a left hand side and a right hand side, said lower edge including a foldable flap, said flaps foldable over the end sectors and the median panel.

11. The tool belt of claim 1 wherein at least one of said left hand segment and said right hand segment is comprised of multiple layers of flexible air permeable materials, including an outside layer, an inside layer and at least one intermediate layer, said intermediate layer including throughpassages for air flow.

12. The tool belt of claim 11 wherein the throughpassage comprises a pattern of throughpassages adjacent the lower edge margin.

13. The tool belt of claim 12 wherein the array of throughpassages is formed with an arcuate boundary and the lower edge margin.

14. The tool belt of claim 1 wherein at least one of said left hand segments and said right hand segments is comprised of multiple layers of flexible materials including an inside layer, at least one intermediate layer and an outside layer, said layers having a generally congruent boundary and joined together about their boundary,
the intermediate layer having a portion thereof excised, said inside and outside layers having air flow passages over the excised portion to allow air access to a person wearing a belt on their waist.

15. The tool belt of claim 1 wherein at least one of said left hand and right hand segments is comprised of multiple layers of joined material having air flow passages through the layers.

16. The tool belt of claim 15 wherein the air flow passages are adjacent the lower edge margin.

17. A multiple component tool belt comprising, in combination:
a left hand belt segment;
a separate substantially mirror image right hand belt segment; and
a separate center belt segment connectable simultaneously to the left hand segment and the right hand segment,
said left and right hand segments each including a buckle end and an opposite center connection end,
the center connection ends comprised of a plurality of removable sections, each removable section upon removal from the segment effecting shortening of the segment and effective shortening of the assembled tool belt comprised of the left hand, center and right hand segments joined together, said connection ends and center segment including a fastener mechanism for releasably joining the segments.

18. The tool belt of claim 17 wherein at least one of the segments includes air passages therethrough.

19. The tool belt of claim 17 wherein the center segment includes flaps for fitting over the center connection ends of the left hand segment and right hand segment.

20. The tool belt of claim 17 wherein each of the removable sections are separated from each other by a boundary for cutting the section from the tool belt segment.
